SEQUENCE LISTING (106 sequences)

SEQ ID NO: 1 - protein, 4 aa, Artificial Sequence
  Synthetic  peptide
FEFE

SEQ ID NO: 2 - protein, 4 aa, Artificial Sequence
  Synthetic peptide
FEFK

SEQ ID NO: 3 - protein, 4 aa, Artificial Sequence
  Synthetic peptide
FEFD

SEQ ID NO: 4 - protein, 4 aa, Artificial Sequence
  Synthetic peptide
FEFR

SEQ ID NO: 5 - protein, 4 aa, Artificial Sequence
  Synthetic peptide
FRFR

SEQ ID NO: 6 - protein, 4 aa, Artificial Sequence
  Synthetic peptide
FRFK

SEQ ID NO: 7 - protein, 4 aa, Artificial Sequence
  Synthetic peptide
FRFE

SEQ ID NO: 8 - protein, 4 aa, Artificial Sequence
  Synthetic peptide
FRFD

SEQ ID NO: 9 - protein, 4 aa, Artificial Sequence
  Synthetic peptide
FKFE

SEQ ID NO: 10 - protein, 4 aa, Artificial Sequence
  Synthetic peptide
FKFK

SEQ ID NO: 11 - protein, 4 aa, Artificial Sequence
  Synthetic peptide
FKFR

SEQ ID NO: 12 - protein, 4 aa, Artificial Sequence
  Synthetic peptide
FKFD

SEQ ID NO: 13 - protein, 4 aa, Artificial Sequence
  Synthetic peptide
FDFD

SEQ ID NO: 14 - protein, 4 aa, Artificial Sequence
  Synthetic peptide
FDFE

SEQ ID NO: 15 - protein, 4 aa, Artificial Sequence
  Synthetic peptide
FDFR

SEQ ID NO: 16 - protein, 4 aa, Artificial Sequence
  Synthetic peptide
FDFK

SEQ ID NO: 17 - protein, 4 aa, Artificial Sequence
  Synthetic peptide
WEWE

SEQ ID NO: 18 - protein, 4 aa, Artificial Sequence
  Synthetic peptide
WKWK

SEQ ID NO: 19 - protein, 4 aa, Artificial Sequence
  Synthetic peptide
WRWR

SEQ ID NO: 20 - protein, 4 aa, Artificial Sequence
  Synthetic peptide
WEWK

SEQ ID NO: 21 - protein, 4 aa, Artificial Sequence
  Synthetic peptide
WKWE

SEQ ID NO: 22 - protein, 4 aa, Artificial Sequence
  Synthetic peptide
WEWR

SEQ ID NO: 23 - protein, 4 aa, Artificial Sequence
  Synthetic peptide
WRWE

SEQ ID NO: 24 - protein, 4 aa, Artificial Sequence
  Synthetic peptide
WKWR

SEQ ID NO: 25 - protein, 4 aa, Artificial Sequence
  Synthetic peptide
WRWK

SEQ ID NO: 26 - protein, 4 aa, Artificial Sequence
  Synthetic peptide
WDWD

SEQ ID NO: 27 - protein, 4 aa, Artificial Sequence
  Synthetic peptide
WDWE

SEQ ID NO: 28 - protein, 4 aa, Artificial Sequence
  Synthetic peptide
WEWD

SEQ ID NO: 29 - protein, 4 aa, Artificial Sequence
  Synthetic peptide
WDWK

SEQ ID NO: 30 - protein, 4 aa, Artificial Sequence
  Synthetic peptide
WKWD

SEQ ID NO: 31 - protein, 4 aa, Artificial Sequence
  Synthetic peptide
WDWR

SEQ ID NO: 32 - protein, 4 aa, Artificial Sequence
  Synthetic peptide
WRWD

SEQ ID NO: 33 - protein, 4 aa, Artificial Sequence
  Synthetic peptide
IEIE

SEQ ID NO: 34 - protein, 4 aa, Artificial Sequence
  Synthetic peptide
IEIK

SEQ ID NO: 35 - protein, 4 aa, Artificial Sequence
  Synthetic peptide
IRIR

SEQ ID NO: 36 - protein, 4 aa, Artificial Sequence
  Synthetic peptide
IKIK

SEQ ID NO: 37 - protein, 4 aa, Artificial Sequence
  Synthetic peptide
IKIE

SEQ ID NO: 38 - protein, 4 aa, Artificial Sequence
  Synthetic peptide
IEIR

SEQ ID NO: 39 - protein, 4 aa, Artificial Sequence
  Synthetic peptide
IRIE

SEQ ID NO: 40 - protein, 4 aa, Artificial Sequence
  Synthetic peptide
IKIR

SEQ ID NO: 41 - protein, 4 aa, Artificial Sequence
  Synthetic peptide
IRIK

SEQ ID NO: 42 - protein, 4 aa, Artificial Sequence
  Synthetic peptide
IDID

SEQ ID NO: 43 - protein, 4 aa, Artificial Sequence
  Synthetic peptide
IDFE

SEQ ID NO: 44 - protein, 4 aa, Artificial Sequence
  Synthetic peptide
IEID

SEQ ID NO: 45 - protein, 4 aa, Artificial Sequence
  Synthetic peptide
IDIK

SEQ ID NO: 46 - protein, 4 aa, Artificial Sequence
  Synthetic peptide
IKID

SEQ ID NO: 47 - protein, 4 aa, Artificial Sequence
  Synthetic peptide
IDIR

SEQ ID NO: 48 - protein, 4 aa, Artificial Sequence
  Synthetic peptide
IRID

SEQ ID NO: 49 - protein, 4 aa, Artificial Sequence
  Synthetic peptide
YEYE

SEQ ID NO: 50 - protein, 4 aa, Artificial Sequence
  Synthetic peptide
YKYK

SEQ ID NO: 51 - protein, 4 aa, Artificial Sequence
  Synthetic peptide
YRYR

SEQ ID NO: 52 - protein, 4 aa, Artificial Sequence
  Synthetic peptide
YEYK

SEQ ID NO: 53 - protein, 4 aa, Artificial Sequence
  Synthetic peptide
YKYE

SEQ ID NO: 54 - protein, 4 aa, Artificial Sequence
  Synthetic peptide
YEYR

SEQ ID NO: 55 - protein, 4 aa, Artificial Sequence
  Synthetic peptide
YRYE

SEQ ID NO: 56 - protein, 4 aa, Artificial Sequence
  Synthetic peptide
YKYR

SEQ ID NO: 57 - protein, 4 aa, Artificial Sequence
  Synthetic peptide
YRYK

SEQ ID NO: 58 - protein, 4 aa, Artificial Sequence
  Synthetic peptide
YDYD

SEQ ID NO: 59 - protein, 4 aa, Artificial Sequence
  Synthetic peptide
YDYE

SEQ ID NO: 60 - protein, 4 aa, Artificial Sequence
  Synthetic peptide
YEYD

SEQ ID NO: 61 - protein, 4 aa, Artificial Sequence
  Synthetic peptide
YDYK

SEQ ID NO: 62 - protein, 4 aa, Artificial Sequence
  Synthetic peptide
YKYD

SEQ ID NO: 63 - protein, 4 aa, Artificial Sequence
  Synthetic peptide
YDYR

SEQ ID NO: 64 - protein, 4 aa, Artificial Sequence
  Synthetic peptide
YRYD

SEQ ID NO: 65 - protein, 4 aa, Artificial Sequence
  Synthetic peptide
XEXE

SEQ ID NO: 66 - protein, 4 aa, Artificial Sequence
  Synthetic peptide
XKXK

SEQ ID NO: 67 - protein, 4 aa, Artificial Sequence
  Synthetic peptide
XRXR

SEQ ID NO: 68 - protein, 4 aa, Artificial Sequence
  Synthetic peptide
XEXK

SEQ ID NO: 69 - protein, 4 aa, Artificial Sequence
  Synthetic peptide
XKXE

SEQ ID NO: 70 - protein, 4 aa, Artificial Sequence
  Synthetic peptide
XEXR

SEQ ID NO: 71 - protein, 4 aa, Artificial Sequence
  Synthetic peptide
XRXE

SEQ ID NO: 72 - protein, 4 aa, Artificial Sequence
  Synthetic peptide
XKXR

SEQ ID NO: 73 - protein, 4 aa, Artificial Sequence
  Synthetic peptide
XRXK

SEQ ID NO: 74 - protein, 4 aa, Artificial Sequence
  Synthetic peptide
XDXD

SEQ ID NO: 75 - protein, 4 aa, Artificial Sequence
  Synthetic peptide
XDXE

SEQ ID NO: 76 - protein, 4 aa, Artificial Sequence
  Synthetic peptide
XEXD

SEQ ID NO: 77 - protein, 4 aa, Artificial Sequence
  Synthetic peptide
XDXK

SEQ ID NO: 78 - protein, 4 aa, Artificial Sequence
  Synthetic peptide
XKXD

SEQ ID NO: 79 - protein, 4 aa, Artificial Sequence
  Synthetic peptide
XDXR

SEQ ID NO: 80 - protein, 4 aa, Artificial Sequence
  Synthetic peptide
XRXD

SEQ ID NO: 81 - protein, 8 aa, Artificial Sequence
  Synthetic peptide
FEFKFEFK

SEQ ID NO: 82 - protein, 8 aa, Artificial Sequence
  Synthetic peptide
FEFEFKFK

SEQ ID NO: 83 - protein, 8 aa, Artificial
  Synthetic peptide
FDFKFDFK

SEQ ID NO: 84 - protein, 8 aa, Artificial
  Synthetic peptide
FDFDFKFK

SEQ ID NO: 85 - protein, 8 aa, Artificial
  Synthetic peptide
FEFRFEFR

SEQ ID NO: 86 - protein, 8 aa, Artificial
  Synthetic peptide
FEFEFRFR

SEQ ID NO: 87 - protein, 8 aa, Artificial
  Synthetic peptide
YDYKYDYK

SEQ ID NO: 88 - protein, 8 aa, Artificial
  Synthetic peptide
YDYDYKYK

SEQ ID NO: 89 - protein, 8 aa, Artificial
  Synthetic peptide
YEYRYEYR

SEQ ID NO: 90 - protein, 8 aa, Artificial
  Synthetic peptide
YEYKYEYK

SEQ ID NO: 91 - protein, 8 aa, Artificial
  Synthetic peptide
YEYEYKYK

SEQ ID NO: 92 - protein, 8 aa, Artificial
  Synthetic peptide
WEWKWEWK

SEQ ID NO: 93 - protein, 8 aa, Artificial
  Synthetic peptide
WEWEWKWK

SEQ ID NO: 94 - protein, 8 aa, Artificial
  Synthetic peptide
WDWKWDWK

SEQ ID NO: 95 - protein, 8 aa, Artificial
  Synthetic peptide
WDWDWKWK

SEQ ID NO: 96 - protein, 8 aa, Artificial Sequence
  Synthetic peptide
FEFKFEFK

SEQ ID NO: 97 - protein, 8 aa, Artificial Sequence
  Synthetic peptide
FEFKFEFK

SEQ ID NO: 98 - protein, 8 aa, Artificial Sequence
  Synthetic peptide
FEFKFEFK

SEQ ID NO: 99 - protein, 8 aa, Artificial Sequence
  Synthetic peptide
FEFKFEFK

SEQ ID NO: 100 - protein, 8 aa, Artificial Sequence
  Synthetic peptide
FEFKFEFK

SEQ ID NO: 101 - protein, 8 aa, Artificial Sequence
  Synthetic peptide
FEFKFEFK

SEQ ID NO: 102 - protein, 8 aa, Artificial Sequence
  Synthetic peptide
FEFKFEFK

SEQ ID NO: 103 - protein, 8 aa, Artificial Sequence
  Synthetic peptide
FEFKFEFK

SEQ ID NO: 104 - protein, 8 aa, Artificial Sequence
  Synthetic peptide
FEFKFEFK

SEQ ID NO: 105 - protein, 8 aa, Artificial Sequence
  Synthetic peptide
FEFKFEFK

SEQ ID NO: 106 - protein, 8 aa, Artificial Sequence
  Synthetic peptide
FEFKFEFK

(19) United States
(12) Patent Application Publication
(10) Pub. No.: US 2015/0307835 A1
(43) Pub. Date: Oct. 29, 2015

(54) PEPTIDES COMPRISING A SHORT-CHAIN POLYETHYLENE GLYCOL MOIETY

(71) Applicant: Peptisyntha SA, Brussels (BE)

(72) Inventors: Laurent JEANNIN, Brussels (BE); Wafa MOUSSA, Nice (FR); Martin BOUSMANNE, Etterbeek (BE)

(73) Assignee: Peptisyntha SA, Brussels (BE)

(21) Appl. No.: 14/419,122

(22) PCT Filed: Jul. 29, 2013
(86) PCT No.: PCT/EP2013/065876

§ 371 Date: Feb. 2, 2015

(30) Foreign Application Priority Data
Aug. 1, 2012 (EP) 12178783.2
Nov. 9, 2012 (EP) 12192025.0

(51) Int. Cl. C12N 5/00; A61K 38/06; A61K 47/48; A61K 47/42; A61K 9/06

(52) U.S. Cl. CPC C12N 5/0068; A61K 47/42; A61K 9/06; A61K 47/48238; A61K 38/06; C12N 2533/30; C12N 2533/50; C12N 2539/00

(57) ABSTRACT

Described are compounds comprising a peptide moiety linked through a linkage to a short-chain polyethylene glycol moiety, more particularly compounds wherein the peptide moiety comprises a self-assembling peptide sequence, and compositions and hydrogels comprising these compounds. Further, methods for the preparation of the present compounds comprising a peptide moiety linked through a linkage to a short-chain polyethylene glycol moiety are disclosed.

This application claims priority to European application No. 12178783.2 filed 1 Aug. 2012 and European application No. 12192025.0 filed 9 Nov. 2012, the whole content of these applications being incorporated herein by reference for all purposes.

The present invention relates to compounds comprising a peptide moiety linked through a linkage to a short-chain polyethylene glycol moiety, more particularly it relates to compounds wherein the peptide moiety comprises a self-assembling peptide sequence, and to compositions comprising these compounds. Further, the invention relates to methods for the preparation of the present compounds comprising a peptide moiety linked through a linkage to a short-chain polyethylene glycol moiety.

Self-assembling peptides comprising alternating hydrophobic and hydrophilic amino acids that self-assemble into a macroscopic structure have been reported. D. G. Osterman et al. described peptides designed to faun amphiphilic β-strand or β-sheet structures (Journal of Cellular Biochemistry, vol. 29, p. 57-72, 1985). Self-assembling peptides are able to form hydrogels which can serve as matrix for various cell-based applications.

Despite the availability of peptide hydrogels, there is a continuous need for improved hydrogels providing new, or improved, applications in, for example, the fields of drug delivery or cell and tissue culture.

Accordingly, it is an object of the present invention, amongst other objects, to provide compounds that can be used in the preparation of a hydrogel suitable for cell and tissue culture with, for example, improved cell adhesion and cell growth.

Further, it is an object of the present invention, amongst other objects, to provide compounds that can be used for providing a hydrogel mimicking the extracellular matrix (ECM) and/or being more biocompatible and/or being less toxic.

Furthermore, it is an object of the present invention, amongst other objects, to provide compounds that can be used for providing a hydrogel facilitating, for example, the harvesting of cultured cells in a more economical and practical way.

Still further, it is an object of the present invention, amongst other objects, to provide compounds with an improved aqueous solubility and/or an improved wettability.

Yet another object of the present invention, amongst other objects, is to provide compounds with improved properties that may be suitable as a vehicle for the formulation of cells, e.g. in cell therapy, and/or that may be applicable for parenteral application. Furthermore, the compounds might show improved viscosity and/or improved rheological behavior.

Another object of the present invention, amongst other objects, is to provide compounds with improved properties for coating of surfaces of vessels used for cell applications. Advantageously, the compounds and/or the hydrogels containing them show an improved fixation to the surface of the vessel. Also advantageously, they lead to a reduction in cost for the coating application.

The above objects, amongst other objects, are met at least partially, if not completely, by one or more embodiments of this invention.

Accordingly, one embodiment of the invention relates to a compound comprising a peptide moiety linked through a linkage to a short-chain polyethylene glycol moiety.

### BRIEF DESCRIPTION OF THE DRAWINGS

FIG. 1 shows the ¹H NMR spectrum of the product according to example 4 (SEQ ID NO:100).

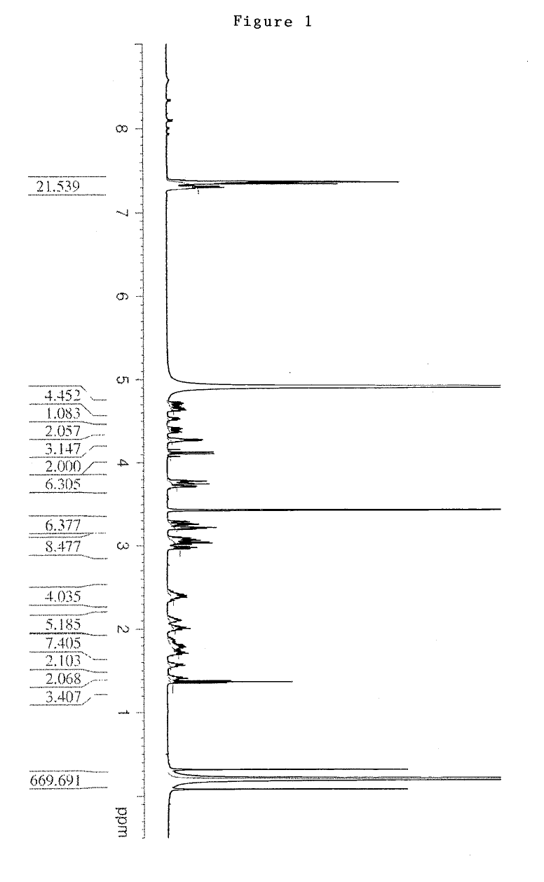

FIG. 2 shows the ¹H NMR spectrum of the product according to example 7 (SEQ ID NO:103).

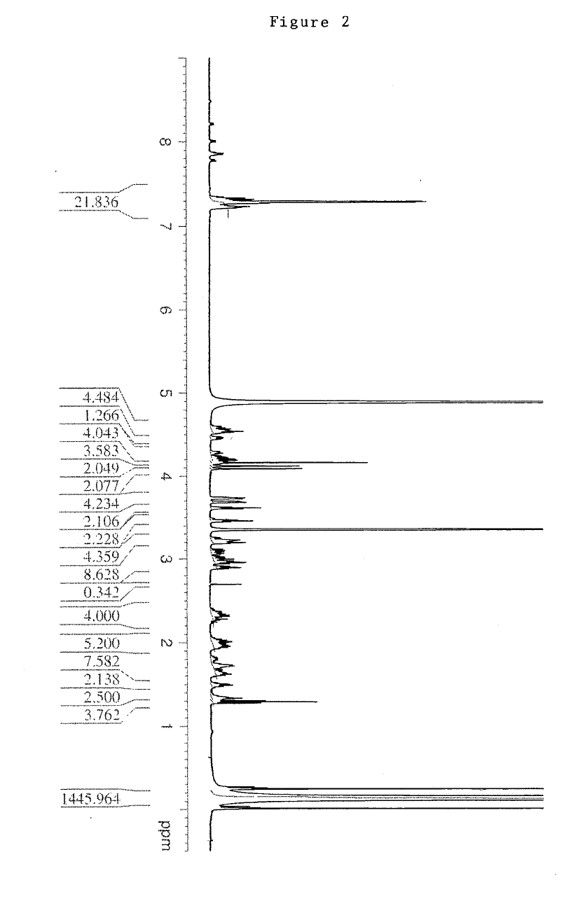

As used herein, the term “short-chain polyethylene glycol” refers to an oligomer of oxyethylene according to the formula:

HOCH₂(CH₂—O—CH₂—)CH₂O—

wherein n is 0 to 24. As used herein, the term “oxyethylene” refers to the moiety —OCH₂CH₂—. Thus, the short-chain polyethylene glycol moiety comprises equal to or less than 25 oxyethylene moieties. In a preferred embodiment the short-chain polyethylene glycol moiety comprises equal to or less than 10 oxyethylene moieties, more preferably equal to or less than 5 oxyethylene moieties. Most preferably, the short-chain polyethylene glycol moiety comprises from 1 to 5 oxyethylene moieties.

In another preferred embodiment the short-chain polyethylene glycol moiety comprises one or more functional groups.

As used herein, “functional group” is intended to denote atoms or small groups of atoms that have special chemical properties and which define the chemistry of an organic compound. Examples of functional groups include alkenyl, alkynyl, (hetero)aryl, hydroxyl, amine, ammonium, carbonyl, carboxyl, phosphate, sulphate, carboxamide, carbonate, carbamate, urea, ester, nitrate, nitro and nitrile. The functional group can be linked to the short-chain polyethylene glycol moiety directly, i.e. through a chemical bond; or indirectly through a linking group. Suitable linking groups are given below. More preferred are functional groups that can be protonated or deprotonated under physiological conditions. As used herein, the term “physiological conditions” is intended to denote conditions of the external or internal milieu that may occur in nature for organisms or cell cultures. A temperature range of 20-40° C. and pH of 6-8 are examples of physiological conditions as used herein. Most preferably, the functional group is selected from the group consisting of NH₂ and COOH.

As used herein, “linkage” is intended to denote any means of covalently connecting two different moieties within a compound of the invention. In a preferred embodiment such linkage can be a direct linkage, i.e. a covalent bond between the atoms of the peptide moiety and the atoms of the short-chain polyethylene glycol moiety. Alternatively, the linkage can be indirect, i.e., through a linking group. Examples of suitable linking groups are a linear or branched alkylidene, especially a polymethylene group comprising 1 to 10 carbon atoms;
a thioether linkage, preferably according to the formula:

—[C(O)]_{z}—(CH₂)_{w}—S(CH₂)ₓ—[C(O)]_{y}—
wherein w and x independently are 0-10, preferably 1, 2, 3 or 4; y and z independently are 0 or 1;
an amino linkage, preferably according to the formula:

—[C(O)]_{z}—(CH₂)_{w}—NH—(CH₂)ₓ—[C(O)]_{y}—
wherein w and x independently are 0-10, preferably 1, 2, 3 or 4; y and z independently are 0 or 1;
an amido linkage, preferably according to the formula:

—C(O)—NH—(CH₂)ₓ—[C(O)]_{y}—
wherein x is 0-10, preferably x is 1, 2, 3, 4 or 5, more preferably x is 1, 2, 3 or 4; y is 0 or 1;
an ester linkage, preferably according to the formula:

—C(O)—O—(CH₂)ₓ—[C(O)]_{y}—
wherein x is 1-10, preferably x is 1, 2, 3 or 4; y is 0 or 1;
or an ether linkage, preferably according to the formula:

—[C(O)]_{z}—(CH₂)_{w}—O—(CH₂)ₓ—[C(O)]_{y}—
wherein w and x independently are 0-10, preferably 1, 2, 3 or 4; y and z independently are 0 or 1.

In a more preferred embodiment, the linkage is —C(O)CH₂OCH₂C(O)—, —C(O)CH₂CH₂C(O)— or —C(O)CH₂CH₂CH₂C(O)—.

As used herein, the term “peptide moiety” comprises peptides and peptide analogues. Peptide analogues comprise natural amino acids and non-natural amino acids. They can also comprise modifications such as ester or amide formation on the C-terminus or N-terminus, respectively. An example for a modification on the C-terminus is the introduction of an ethyl ester which can be accomplished by methods known in the art. All amino acids can be either the L- or D-isomer. The peptides or peptide analogues can also comprise amino acid mimetics that function in a manner similar to the naturally occurring amino acids. The peptides may also be formed from amino acids analogues that have modified R groups or modified peptide backbones. Peptide analogues usually include at least one bond in the peptide sequence which is different from an amide bond, such as urethane, urea, ester or thioester bond. Peptides or peptide analogues according to the present invention can be linear, cyclic or branched and are preferably linear.

As used herein, the term “amino acid” (Xaa) is intended to denote any compound comprising at least one NR₁R₂ group, preferably at least one NH₂ group, and at least one carboxyl group. The amino acids of this invention can be naturally occurring or synthetic. The natural amino acids, with exception of glycine, contain a chiral carbon atom. Unless otherwise specifically indicated, the compounds containing natural amino acids with the L-configuration are preferred. The amino acids can be selected from, for example, β-alanine, γ-aminobutyric acid, 5-aminovaleric acid, glycine, phenylglycine, homoarginine, alanine, valine, norvaline, leucine, norleucine, isoleucine, serine, isoserine, homoserine, threonine, allothreonine, methionine, ethionine, glutamic acid, aspartic acid, asparagine, cysteine, cystine, phenylalanine, tyrosine, tryptophan, lysine, hydroxylysine, arginine, histidine, ornithine, glutamine, citrulline, proline, and 4-hydroxyproline. Amino acid residues are abbreviated as follows throughout the application: Alanine is Ala or A; β-Alanine is β-Ala; γ-aminobutyric acid is GABA; 5-aminovaleric acid is Ava; Arginine is Arg or R; Homoarginine is Har or hR; Alanine is Ala or A; Asparagine is Asn or N; Aspartic acid is Asp or D; Cysteine is Cys or C; Glutamic acid is Glu or E; Glutamine is Gln or Q; Glycine is Gly or G; Histidine is His or H; Homoserine is Hse; Hydroxylysine is Hyl; Isoleucine is Ile or I; Leucine is Leu or L; Lysine is Lys or K; Methionine is Met or M; Norleucine is Nle; Ornithine is Orn; Phenylalanine is Phe or F; Proline is Pro or P; 4-Hydroxyproline is Hyp or O; Serine is Ser or S; Threonine is Thr or T; Tryptophan is Trp or W; Tyrosine is Tyr or Y; Valine is Val or V.

In a preferred embodiment the peptide moiety is able to self-assemble in a β-sheet, a coiled coil α-helix structure, a peptide triple helix structure, or combinations thereof. Self-assembling amino acid sequences capable of assembling into a β-sheet are more preferred.

According to another preferred embodiment of the present invention, the peptide moiety is an octapeptide moiety comprising alternating hydrophobic and charged amino acids. Hydrophobic amino acids are often selected from the group consisting of Phenylalanine (Phe or F), Tryptophan (Trp or W), Tyrosine (Tyr or Y), Isoleucine (Ile or I), Alanine (Ala or A), Leucine (Leu or L), Valine (Val or V), and Norleucine (Nle); in particular from Phenylalanine (Phe or F), Tryptophan (Trp or W), Tyrosine (Tyr or Y), Isoleucine (Ile or I), and Norleucine (Nle). Charged amino acids are usually selected from the group consisting of Arginine (Arg or R), Aspartic acid (Asp or D), Glutamic acid (Glu or E), Lysine (Lys or K), and Histidine (His or H); particularly from Arginine (Arg or R), Aspartic acid (Asp or D), Glutamic acid (Glu or E), and Lysine (Lys or K).

According to another preferred embodiment of the present invention the octapeptides comprise one type of hydrophobic amino acid and two types of charged amino acids. Especially suitable octapeptides are formed by the combination of two sequences chosen independently from the group consisting of FEFE (SEQ ID NO:1), FEFK (SEQ ID O:2), FEFD (SEQ ID NO:3), FEFR (SEQ ID NO:4), FRFR (SEQ ID NO:5), FRFK (SEQ ID NO:6), FRFE (SEQ ID NO:7), FRFD (SEQ ID NO:8), FKFE (SEQ ID NO:9), FKFK (SEQ ID NO:10), FKFR (SEQ ID NO:11), FKFD (SEQ ID NO:12), FDFD (SEQ ID NO:13), FDFE (SEQ ID NO:14), FDFR (SEQ ID NO:15), FDFK (SEQ ID NO:16), WEWE (SEQ ID NO:17), WKWK (SEQ ID NO:18), WRWR (SEQ ID NO:19), WEWK (SEQ ID NO:20), WKWE (SEQ ID NO:21), WEWR (SEQ ID NO:22), WRWE (SEQ ID NO:23), WKWR (SEQ ID NO:24), WRWK (SEQ ID NO:25), WDWD (SEQ ID NO:26), WDWE (SEQ ID NO:27), WEWD (SEQ ID NO:28), WDWK (SEQ ID NO:29), WKWD (SEQ ID NO:30), WDWR (SEQ ID NO:31), WRWD (SEQ ID NO:32), IEIE (SEQ ID NO:33), IEIK (SEQ ID NO:34), IRIR (SEQ ID NO:35), IKIK (SEQ ID NO:36), IKIE (SEQ ID NO:37), IEIR (SEQ ID NO:38), IRIE (SEQ ID NO:39), IKIR (SEQ ID NO:40), IRIK (SEQ ID NO:41), IDID (SEQ ID NO:42), IDFE (SEQ ID NO:43), IEID (SEQ ID NO:44), IDIK (SEQ ID NO:45), IKID (SEQ ID NO:46), IDIR (SEQ ID NO:47), IRID (SEQ ID NO:48), YEYE (SEQ ID NO:49), YKYK (SEQ ID NO:50), YRYR (SEQ ID NO:51), YEYK (SEQ ID NO:52), YKYE (SEQ ID NO:53), YEYR (SEQ ID NO:54), YRYE (SEQ ID NO:55), YKYR (SEQ ID NO:56), YRYK (SEQ ID NO:57), YDYD (SEQ ID NO:58), YDYE (SEQ ID NO:59), YEYD (SEQ ID NO:60), YDYK (SEQ ID NO:61), YKYD (SEQ ID NO:62), YDYR (SEQ ID NO:63), YRYD (SEQ ID NO:64), Nle-E-Nle-E (SEQ ID NO:65), Nle-K-Nle-K (SEQ ID NO:66), Nle-R-Nle-R (SEQ ID NO:67), Nle-E-Nle-K (SEQ ID NO:68), Nle-K-Nle-E (SEQ ID NO:69), Nle-E-Nle-R (SEQ ID NO:70), Nle-R-Nle-E (SEQ ID NO:71), Nle-K-Nle-R (SEQ ID NO:72), Nle-R-Nle-K (SEQ ID NO:73), Nle-D-Nle-D (SEQ ID NO:74), Nle-D-Nle-E (SEQ ID NO:75), Nle-E-Nle-D (SEQ ID NO:76), Nle-D-Nle-K (SEQ ID NO:77), Nle-K-Nle-D (SEQ ID NO:78), Nle-D-Nle-R (SEQ ID NO:79), and Nle-R-Nle-D (SEQ ID NO:80). The two sequences can be the same or different, especially the same.

According to yet another preferred embodiment of the present invention the octapeptide moiety might for instance be selected from the group consisting of FEFKFEFK (SEQ ID NO:81), FEFEFKFK (SEQ ID NO:82), FDFKFDFK (SEQ ID NO:83), FDFDFKFK (SEQ ID NO:84), FEFRFEFR (SEQ ID NO:85), FEFEFRFR (SEQ ID NO:86), YDYKYDYK (SEQ ID NO:87), YDYDYKYK (SEQ ID NO:88), YEYRYEYR (SEQ ID NO:89), YEYKYEYK (SEQ ID NO:90), YEYEYKYK (SEQ ID NO:91), WEWKWEWK (SEQ ID NO:92), WEWEWKWK (SEQ ID NO:93), WDWKWDWK (SEQ ID NO:94), WDWDWKWK (SEQ ID NO:95). Most preferably the amino sequence is FEFKFEFK (SEQ ID NO:81).

In yet another preferred embodiment the peptide moiety is linked through a linkage to the short-chain polyethylene glycol moiety at the terminus of the peptide moiety, More preferably, it is linked through a linkage at the C-terminus of the peptide moiety.

Another embodiment of the invention relates to a composition comprising a compound according to the invention as described above and further comprising at least one solvent and/or comprising at least one additive. Examples for suitable solvents are water, ethanol, methanol, isopropanol, propanol, butanol, acetonitrile, acetone, dimethylsulfoxide, N-methylpyrrolidinone, N,N-dimethylformamide, N,N-dimethylacetamide, or mixtures thereof. Especially suitable is water or mixtures of water with at least one further solvent. Examples for suitable additives for cell culture are nutrients, antibiotics, buffers, and/or growth factors. Examples for suitable buffers are Hank's Balanced Salt Solution (HBSS) and Dulbecco's Modified Eagle Medium (DMEM).

Preferably, the composition is a pharmaceutical composition comprising a compound according to the invention. The pharmaceutical composition may be administered parenterally, for example, intravenously, intraarterially, intraperitoneally, intrathecally, intraventricularly, intraurethrally, intrasternally, intracranially, intramuscularly or subcutaneously, or they may be administered using needleless injection techniques, For such parenteral administration a sterile aqueous solution which may contain other substances, for example, enough salts or glucose to make the solution isotonic with blood may be used. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art. Preferably, the pharmaceutical composition comprises a pharmaceutically active ingredient, more preferably the pharmaceutically active ingredient is a peptide. One example of a suitable pharmaceutical active ingredient is the pituitary GnRH-receptor agonist Leuprolide.

In another preferred embodiment, the composition further comprises a further peptide. An example of a suitable further peptide is a peptide that is able to self-assemble. An especially suitable further peptide comprises the same sequence as the peptide moiety of the compound of the invention comprised in the composition.

As indicated above, the compounds comprising a peptide moiety linked through a linkage to a short-chain polyethylene glycol moiety according to the invention are capable of providing a hydrogel suitable for cell and tissue culture.

Accordingly, another embodiment of the invention are hydrogels comprising a compound or a composition according to this invention as described above.

Another embodiment concerns hydrogels comprising at least one compound according to this invention as described above.

As used herein, the term “hydrogel” is intended to denote a network of polymer chains in which water is used as a dispersion medium.

Preferably, the hydrogel further comprises at least one self-assembling peptide linked through a linkage to a bioactive sequence, even more preferably the self-assembling peptide is linked through a linkage to a sequence selected from RGD (Arg-Gly-Asp) and hRGD (Har-Gly-Asp), especially the hydrogel further comprises FEFKFEFK (SEQ ID NO:81) linked to the bioactive sequence hRGD through a covalent bond. The linkage can be the same as defined above.

The term “bioactive sequence” is intended to denote an amino acid sequence which has a specific biological function, e.g. the promotion of cell adhesion, cell growth and/or cell differentiation,

Examples of suitable self-assembling peptides linked through a linkage to a bioactive sequence are disclosed in WO 2012/045824, which is hereby fully incorporated by reference.

In yet another embodiment the hydrogel further comprises at least one self-assembling peptide linked through a linkage to a polyacrylamide moiety. Preferably, the self-assembling peptides linked through a linkage to a polyacrylamide moiety comprises an oligopeptide moiety comprising alternating hydrophobic and charged amino acids, more preferably the oligopeptide moiety is selected from the group consisting of SEQ ID NO:81 to SEQ ID NO:95, especially the peptide moiety is SEQ ID NO:81. Preferably, the polyacrylamide moiety is selected from the group consisting of poly-(N-isopropylacrylamide) (pIPA), 2-hydroxyethyl methacrylate (pHEMA) and poly[N-(2-hydroxypropyl)methacrylamide (pHPMA), more preferably the polyacrylamide moiety is poly[N-(2-hydroxypropyl)methacrylamide (pHPMA). The molecular weight of the polyacrylamide moiety is preferably from 2500 to 25000 Dalton, more preferably from 5000 to 10000 Dalton. The linkage between the polyacrylamide and the peptide moiety can be the same as defined above. Especially suitable linkages are a covalent bond, beta-alanine, or aminovaleric acid. Most preferably, the self-assembling peptide linked through a linkage to a polyacrylamide residue according to this embodiment is a peptide of SEQ ID NO:81 linked through a beta-alanine moiety to poly[N-(2-hydroxypropyl)methacrylamide (pHPMA),

In still another embodiment the hydrogel further comprises at least two further peptides wherein the at least two further peptides are at least one self-assembling peptide linked to a bioactive sequence and at least one self-assembling peptide linked through a linkage to a polyacrylamide moiety.

In an alternative embodiment, the hydrogel comprises at least one self-assembling peptide linked through a linkage to a bioactive sequence and at least one self-assembling peptide linked through a linkage to a polyacrylamide moiety. Suitable peptides in this embodiment are as defined above.

Advantageously, all peptides comprised in the hydrogels according to the invention comprise the same peptide sequence in the peptide moiety.

The preparation of a hydrogel according to the invention may comprise the steps of:
a) adding at least water to a compound or to a composition according to the invention;
b) optionally adding a further peptide that is able to self-assemble;
c) optionally adjusting the pH and/or the ionic strength of the resulting medium, with or without the further peptide that is able to self-assemble, to form a hydrogel.

Step a) of the method for preparing a hydrogel may further comprise adding at least one further suitable solvent. Examples of suitable solvents or solvent mixtures are given above. The hydrogels thus obtained show thixotropic behavior suitable for therapeutic and/or cell culture applications.

The surface of vessels used for cell culture applications can be coated to improve adhesion, proliferation and/or growth of the cells. For example, collagen has been reported for this purpose. Disadvantages associated with the use of collagen in cell culture applications include the high cost of pure collagen and a variability of the material, e.g. in twins of crosslink density, fibre size and/or trace impurities. Accordingly, another embodiment of the present invention relates to the use of the compounds and the hydrogels of the present invention for coating of a surface used in cell culture. Examples of preferred surfaces are surfaces being part of a cell culture insert, a tissue culture flask, a Petri dish, a multi-well plate or a bioreactor. Vessels of the before-mentioned kind can all be commercially obtained, for example from EMD Millipore Corporation. The surfaces can be made from different materials, e.g. from polystyrene, polyethylene, polycarbonate, polyurethane, or glass. The surface can advantageously be pretreated by laser etching or chemical etching.

According to another aspect, the present invention relates to methods for preparing the present compounds comprising a peptide moiety linked through a linkage to a short-chain polyethylene glycol moiety. The short-chain polyethylene glycol moiety optionally comprising a functional group and/or the linkages according to the present invention may be coupled to the peptide moiety using techniques known to the skilled man and may proceed via compounds such as mercapto propionic acid, gamma-amino butyric acid, epsilon-amino caproic acid, 3-aminopropionic acid, 5-amino valeric acid, 11-amino undecanoic acid, 8-amino-3,6-dioxaoctanoic acid, succinic anhydride, glutaric anhydride, diglycolic anhydride, or 1-amino-4.7.10-trioxa-13-tridecanamine. The functional groups present in the coupling partners may be suitably protected during the coupling step between the coupling partners. Protecting groups are known in the art of peptide synthesis. Examples of suitable protecting groups include carboxybenzyl (Z) or tert-butyloxycarbonyl (Boc) for an amine and esters such as a tert-butyl ester for a carboxyl group. Protection and deprotection may be performed as known in the art, for example by hydrogenation or acidic cleavage, e.g. using trifluoroacetic acid (TFA). The coupling may be facilitated by using coupling reagents. Examples of coupling reagents include 1-hydroxybenzotriazole, 1-hydroxy-7-azabenzotriazole, N,N′-dicyclohexylcarbodiimide, N,N′-diisopropylcarbodiimide and 1-ethyl-3-(3-dimethylaminopropyl)carbodiimide. Examples for solvents suitable in these reactions include acetonitrile, acetone, dimethylsulfoxide, N-methylpyrrolidinone, N,N-dimethylformamide, N,N-dimethylacetamide, or mixtures thereof.

Should the disclosure of any patents, patent applications, and publications which are incorporated herein by reference conflict with the description of the present application to the extent that it might render a term unclear, the present description shall take precedence.

The present invention is further illustrated below without limiting the scope thereto.

### EXAMPLES

### General Methods:

HPLC: Analyses were performed on an Agilent 1100 series HPLC using “method CH-GS-10”: Column: Merck Chromolith RP C18-e (100 mm×4.6 mm); mobile phase A: water+0.1% TFA, mobile phase B: acetonitrile+0.1% TFA; stoptime: 10.5 min; posttime: 1.0 min; temperature: 40° C.; flow rate: 4.0 ml/min.

### Gradient:

| Time (min) | % solvent B |
|---|---|
| 0.00 | 2.0 |
| 10.00 | 91.1 |
| 10.10 | 100.0 |
| 10.50 | 100.0 |
| 10.60 | 2.0 |

¹H NMR: Bruker AVANCE, 500 MHz; solvent: CD₃OD+2-3 drops TFA; internal standard: octamethycyclotetrasiloxane.

### Example 1

### Z-Phe-Glu(OtBu)-Phe-Lys(Boc)-Phe-Glu(OtBu)-Phe-Lys(Boc)-OEt (SEQ ID NO:97)

50 g Z-Phe-Glu(OtBu)-Phe-Lys(Boc)-Phe-Glu(OtBu)-Phe-Lys(Boc)-OH (SEQ ID NO:96) (32 mmol) and 5.3 g caesium carbonate (16 mmol) were added to 500 ml N,N-dimethylfounamide. 5.5 ml iodoethane (69 mmol) was added and the solution was stirred at 48° C. for 2 h. After filtration and partial evaporation of the N,N-dimethylformamide, the concentrate was poured into 500 ml 2.5 wt.-% aqueous KHSO₄, filtered, washed with water and finally with 500 ml warm ethanol. After drying under reduced pressure at 45° C., 47 g (84%) of a solid was obtained. The HPLC analysis of the product was carried out as follows: a sample was dissolved in 1 ml N,N-dimethylacetamide and analysed using method CH-GS-10. Product purity: 97% of surface area (HPLC).

### Example 2

### H-Phe-Glu(OtBu)-Phe-Lys(Boc)-Phe-Glu(OtBu)-Phe-Lys(Boc)-OEt (SEQ ID NO: 98)

21.6 g Z-Phe-Glu(OtBu)-Phe-Lys(Boc)-Phe-Glu(OtBu)-Phe-Lys(Boc)-OEt (SEQ ID NO:97) (13.6 mmol) was dissolved in 215 ml N,N-dimethylacetamide. After flushing the solution several times with nitrogen gas, 14.5 g Pd/SiO₂ (2 wt.-%) was added and subsequently, hydrogen gas was introduced, After stirring for 2 h, the suspension was passed through a 0.45 μm filter and the Pd/SiO₂ was washed with N,N-dimethylacetamide. The combined filtrates can be used without further purification in the subsequent step. The yield was quantitative.

### Example 3

### Boc-HNCH₂CH₂OCH₂CH₂OCH₂C(O)Phe-Glu(OtBu)-Phe-Lys(Boc)-Phe-Glu(OtBu)-Phe-Lys(Boc)-OEt (SEQ ID NO:99)

1.0 g 8-(t-Butyloxycarbonyl-amino)-3,6-dioxaoctanoic acid (Boc-O2OC—OH.DCHA) (2.25 mmol) was suspended in 50 ml CH₂Cl₂. This suspension was washed with 60 ml 5% aqueous NaCl solution containing 0.43 g KHSO₄ (3.15 mmol), then with 60 ml 5% aqueous NaCl solution and finally with 60 ml distilled water. After concentration in vacuo and azeotropic drying, a sample of the concentrated solution was titrated with 0.1 N aqueous NaOH solution. A N,N-dimethylacetamide solution of H-Phe-Glu(OtBu)-Phe-Lys(Boc)-Phe-Glu(OtBu)-Phe-Lys(Boc)-OEt (SEQ ID NO:98) (1.25 mmol, 20 ml) was added to the concentrated solution of neutralized Boc-O2OC—OH (1.2 mmol). 1-Ethyl-3-(3-dimethylaminopropyl)carbodiimide (0.2 g, 1.3 mmol) and N-hydroxybenzotriazole (0.18 g, 1.3 mmol) were added at room temperature. After HPLC control of the completion of the reaction, the reaction mixture was poured into 200 ml 2.5% aqueous KHSO₄ solution. The resulting precipitate was washed with 25 ml distilled water. After drying in vacuo at 45° C., 1.58 g (1 mmol) of an off-white solid was obtained. The HPLC analysis of the product was carried out as follows: a sample was dissolved in 1 ml N,N-dimethylacetamide and analysed using method CH-GS-10; t_{R}=8.9±0.5 min.

### Example 4

### H₂NCH₂CH₂OCH₂CH₂OCH₂C(O)Phe-Glu-Phe-Lys-Phe-Glu-Phe-Lys-OEt (SEQ ID NO:100)

20 ml trifluoroacetic acid, 2 ml triisopropylsilane, 1 ml ethanol and 1 ml water were dissolved in 20 ml CH₂Cl₂. To this solution, 1.5 g (0.9 mmol) BOCHNCH₂CH₂OCH₂CH₂OCH₂C(O)Phe-Glu(OtBu)-Phe-Lys(Boc)-Phe-Glu(OtBu)-Phe-Lys(Boc)-OEt (SEQ ID NO:99) was added at room temperature. After stirring at room temperature for 1 h, the reaction mixture was concentrated in vacuo. The resulting solid was dispersed in 30 ml methyl t-butyl ether and filtered. 1.26 g of an off-white product was obtained. The HPLC analysis of the product was carried out as follows: a sample was dissolved in 1 ml N,N-dimethylacetamide and analyzed using method CH-GS-10; t_{R}=4.1±0.5 min, Product purity: 85% of surface area (HPLC). The product can be purified further by preparative HPLC to obtain a purity≧95% (surface area) and additionally, can optionally be lyophilized.

### Example 5

### Synthesis of HOOC—CH₂—O—CH₂—C(O)-Phe-Glu(OtBu)-Phe-Lys(Boc)-Phe-Glu(OtBu)-Phe-Lys(Boc)-OEt (SEQ ID NO:101)

0.22 g diglycolic anhydride (1.6 mmol) was added to a solution of 35.5 g H-Phe-Glu(OtBu)-Phe-Lys(Boc)-Phe-Glu(OtBu)-Phe-Lys(Boc)-OEt (SEQ ID NO:98) in N,N-dimethylacetamide. After stirring at room temperature for at least one hour, the reaction mixture was partially concentrated under vacuum and then poured into a solution of KHSO₄ (0.43 g) in 150 ml water. The resulting precipitate was filtered, washed with water (50 ml) twice and then dried under vacuum at 45° C. After drying, an off-white product (2.39 g, 95%) was obtained. The HPLC analysis of the product was carried out as follows: a sample was dissolved in 1 ml N,N-dimethylacetamide and analysed using method CH-GS-10; t_{R}=8.3±0.5 min. Product purity: 88% of surface area (HPLC).

### Example 6

### Synthesis of HOOCCH₂OCH₂CH₂OCH₂CH₂NC(O)CH₂OCH₂C(O)-Phe-Glu(OtBu)-Phe-Lys(Boc)-Phe-Glu(OtBu)-Phe-Lys(Boc)-OEt (SEQ ID NO:102)

8.9 g HOOCCH₂OCH₂C(O)-Phe-Glu(OtBu)-Phe-Lys(Boc)-Phe-Glu(OtBu)-Phe-Lys(Boc)-OEt (SEQ ID NO:101) (5.6 mmol), 1.15 ml diisopropylethylamine (5.68 mmol) and 0.47 ml pyridine (5.68 mmol) were dissolved in a mixture of 70 ml N,N-dimethylacetamide and 20 ml dichloromethane. The reaction mixture was cooled to −5° C. (solution n°1). 1.02 g 8-amino-3,6-dioxaoctanoic acid (6.19 mmol) was dispersed in 2.30 ml dichloromethane containing 1.84 g trimethylsilylacetamide (12.4 mmol) (solution n°2). Solution n°2 was stirred at room temperature and then cooled to 10° C. At −5° C., 0.7 g pivaloyl chloride was added to solution n°1. After about 5 min of further stirring, cooled solution n°2 was added to solution n°1. The reaction mixture was allowed to warm to room temperature. After controlling the completion of the reaction by HPLC, 2 ml water was added, the reaction mixture partially concentrated under vacuum and then poured into a solution of KHSO4 (2.5 g) in 500 ml water. The resulting precipitate was filtered, washed with water (50 ml) twice and then dried under vacuum at 45° C. After drying, 9.17 g (94%) of an off-white product was obtained. The HPLC analysis of the product was carried out as follows: a sample was dissolved in 1 ml N,N-dimethylacetamide and analysed using method CH-GS-10; t_{R}=7.8±0.5 min. Product purity: 89% of surface area (HPLC).

### Example 7

### Synthesis of HOOCCH₂OCH₂CH₂OCH₂CH₂NC(O)CH₂OCH₂C(O)-Phe-Glu-Phe-Lys-Phe-Glu-Phe-Lys-OEt (SEQ ID NO:103)

11.44 g HOOCCH₂OCH₂CH₂OCH₂CH₂NC(O)CH₂OCH₂C(O)-Phe-Glu(OtBu)-Phe-Lys(Boc)-Phe-Glu(OtBu)-Phe-Lys(Boc)-OEt (SEQ ID NO:102) was added to 120 ml of a mixture of trifluoroacetic acid and water (95/5) under stirring. After 30 min at room temperature, the reaction mixture was partially concentrated under vaccum, diluted with dichloromethane (20 ml) and concentrated again. This step of concentrating and dilution was repeated several times. The concentrated solution was added to 1000 ml diisopropylether and a precipitate was formed. After filtration, the precipitate was washed twice with 500 ml diisopropylether and dried under vacuum at 45° C. to yield 10.62 g (85%) of an off-white solid. The HPLC analysis of the product was carried out as follows: a sample was dissolved in 1 ml N,N-dimethylacetamide and analysed using method CH-GS-10; t_{R}=4.2±0.5 min. Product purity: 85% of surface area (HPLC). This crude can optionally be purified by HPLC to reach a purity≧95% (surface area) and can optionally be lyophilized.

### Example 8

### Synthesis of Z-Phe-Glu(OtBu)-Phe-Lys(Boc)-Phe-Glu(OtBu)-Phe-Lys(Boc)-NH—(CH₂)₃—O—(CH₂)₂—O—(CH₂)₂—O—(CH₂)₃—NHBoc (SEQ ID NO:104)

0.784 g Z-Phe-Glu(OtBu)-Phe-Lys(Boc)-Phe-Glu(OtBu)-Phe-Lys(Boc)-OH (SEQ ID NO:96) (0.5 mmol) and 0.165 g 1-(t-butyloxycarbonylamino)-4,7,10-trioxa-13-tridecanamine (Boc-TOTA-NH₂) (0.5 mmol) were dissolved in 8 ml N,N-dimethylacetamide at room temperature. After cooling to 0° C., 83 mg 1-ethyl-3-(3-dimethylaminopropyl)carbodiimide (0.6 mmol) and 117 mg N-hydroxybenzotriazole (0.6 mmol) were added to the solution. The solution was stirred at 0° C. for an additional 3 h and then allowed to warm to room temperature. After control of the completion of the reaction by HPLC, the reaction mixture was added to a solution of KHSO₄ (0.1 g) in 80 ml water. The resulting precipitate was washed with 25 ml distilled water twice. After drying under vacuum at 45° C., 820 mg (87%) of an off-white solid was obtained. The HPLC analysis of the product was carried out as follows: a sample was dissolved in 1 ml N,N-dimethylacetamide and analysed using method CH-GS-10; t_{R}=9.2±0.5 min. Product purity: 88% of surface area (HPLC).

### Example 9

### Synthesis of H-Phe-Glu(OtBu)-Phe-Lys(Boc)-Phe-Glu(OtBu)-Phe-Lys(Boc)-NH—(CH₂)₃—O—(CH₂)₂—O—(CH₂)₂—O—(CH₂)₃—NHBoc (SEQ ID NO:105)

820 mg Z-Phe-Glu(OtBu)-Phe-Lys(Boc)-Phe-Glu(OtBu)-Phe-Lys(Boc)-NH—(CH₂)₃—O—(CH₂)₂—O—(CH₂)₂—O—(CH₂)₃—NHBoc (SEQ ID NO:104) (0.4 mmol) was dissolved in 20 ml N,N-dimethylacetamide and stirred at 45° C. After flushing the solution several times with nitrogen gas, 0.47 g Pd/SiO₂ (2 wt.-%) was added. Hydrogen gas was introduced and the reaction was stirred for 2 h. The suspension was passed through a 0.45 μm filter and the Pd/SiO₂ was washed with N,N-dimethylacetamide. The combined filtrates were partially concentrated under vacuum and poured into a solution of NaHCO₃ (0.05 g) in 35 ml water. After filtration, the precipitate was washed several times with water (10 ml each). After drying under vacuum at 45° C., 490 mg (83%) of an off-white solid was obtained. The HPLC analysis of the product was carried out as follows: a sample was dissolved in 1 ml N,N-dimethylacetamide and analysed by the method CH-GS-10; t_{R}=7.2±0.5 min Product purity: 90% of surface area (HPLC).

### Example 10

### Synthesis of H-Phe-Glu-Phe-Lys-Phe-Glu-Phe-Lys-NH—(CH₂)₃—O—(CH₂)₂—O—(CH₂)₂—O—(CH₂)₃—NH₂(SEQ ID NO:106)

490 mg H-Phe-Glu(OtBu)-Phe-Lys(Boc)-Phe-Glu(OtBu)-Phe-Lys(Boc)-NH—(CH₂)₃—O—(CH₂)₂—O—(CH₂)₂—O—(CH₂)₃—NHBoc (SEQ ID NO:105) was added to 5 ml of a mixture of trifluoroacetic acid and water (95/5) under stirring. After 30 min stirring at room temperature, the reaction mixture is partially concentrated under vacuum, diluted with CH₂Cl₂ and concentrated again. This step of concentrating and dilution was repeated several times. The concentrated solution was then poured into 35 ml diisopropylether to obtain a precipitate which was collected. After drying the precipitate under vacuum at 45° C., 420 mg (85%) of an off-white solid was obtained. The HPLC analysis of the product was carried out as follows: a sample was dissolved in 1 ml N,N-dimethylacetamide and analysed using method CH-GS-10; t_{R}=3.5±0.5 min. Product purity: 88% of surface area (HPLC). This crude may be purified further by preparative HPLC to reach a purity of ≧95% (surface area) and optionally lyophilized to obtain the trifluoroacetate salt.

### Example 11

### Hydrogel Preparation

0.81 mg NaHCO₃ was added to 0.1 ml of a dimethylsulfoxide solution of 0.02 g HOOCCH₂OCH₂CH₂OCH₂CH₂NC(O)CH₂OCH₂C(O)-Phe-Glu-Phe-Lys-Phe-Glu-Phe-Lys-OEt (SEQ ID NO:103). The suspension is vigorously shaken. The suspension is diluted with 3.9 ml Hank's Balanced Salt Solution (HBSS) and the mixture was shaken for 3 min. The resulting gel was transferred to a Vivaspin® ultrafiltration spin column with a molecular weight cut-off varying from 3 to 100 kDa, and centrifuged between 12 000 and 4000 G for at least 15 min. The filtrate was discarded. The same volume of fresh culture media was added on top of the gel, and another centrifugation cycle was performed. This operation was repeated at least twice. During the last washing, fresh HBSS was replaced by fresh Dulbecco's Modified Eagle Medium (DMEM). The washed hydrogel was finally conditioned and stored at 10° C. prior use.

### Example 12

### Synthesis of a HMPA-Oligopeptide

8.0 g H-(3-Ala-OH (90 mmol) was added to 46 ml water containing 7.2 g NaOH (180 mmol). The mixture was cooled to 0° C. 9.7 g methacryloyl chloride (90 mmol) was added dropwise whilst maintaining the temperature inside the reaction mixture below 5° C. After complete addition, the reaction mixture was further stirred for 2 h at room temperature. The pH of the reaction mixture was then brought to 1 to 2 by adding 15 ml concentrated HCl diluted with 60 ml water. The aqueous layer was extracted five times with 100 ml ethyl acetate. The organic layers were concentrated under vacuum. The concentrate was diluted with 150 ml ethyl acetate and further concentrated to remove remaining water. The concentrated solution was cooled to 10° C. and kept at this temperature overnight. The crystals were filtered off and washed with 50 ml diisopropryl ether. After drying under vacuum at 45° C., 9.48 g of an off-white solid was obtained.

5.95 g of the product from the previous step (MA-β-Ala-OH, 36 mmol) was dissolved in 108 ml dichloromethane containing 3.67 g N-methyl morpholine (36 mmol). After cooling to −10° C., 4.96 g isobutylchloroformate (36 mmol) in 3.6 ml dichloromethane was added. The reaction mixture was further stirred for 5 min at −10° C. and afterwards 6.21 g N-hydroxy succinimide (54 mmol) and 5.51 g N-methylmorpholine (54 mmol) diluted in 4 ml dichloromethane were added. At the end of the addition, the reaction mixture was further stirred for 1 h at room temperature. After checking completion of the reaction by HPLC, the organic layer was successively washed with 40 ml 5% aqueous NaCl solution containing 2.5% KHSO, 40 ml 5% aqueous NaCl, 40 ml 5% aqueous NaCl containing 0.8 g NaHCO₃ and finally 40 ml water. After partial concentration of the organic layer under vacuum, the residue was dispersed in isopropyl acetate (200 ml in total) and the concentration was continued. The resulting concentrated suspension (28 g) was stirred at 10° C. for at least 2 h, and then filtered. The solid was washed twice with 10 ml isopropyl acetate, then with 20 ml isopropanol. After drying at 45° C. under vacuum, 5.78 g of an off-white product was obtained,

6.55 g 2-hydroxypropylmethacrylamide (45 mmol) and 1.28 g (5 mmol) of the product from the previous step (MA-β-Ala-O-succinimide) were dissolved in 12 g N,N-dimethylacetamide. 0.98 g of the resulting solution was added to 0.41 g AIBN (2.5 mmol) dissolved in 630 mg N,N-dimethylacetamide. This reaction mixture was heated to 70° C. under mechanical stirring.

The rest of the solution was introduced by means of a syringe connected to a pump at a rate of 0.083 ml/min. At the end of the addition, the reaction mixture was further stirred at 70° C. for 8 h. The resulting viscous solution was diluted with 10 ml N,N-dimethylacetamide and then poured into 300 ml acetone under stirring. The suspension was filtered, and the product was washed once with 150 ml acetone and finally with 100 ml methyl tert-butylether. After drying under vacuum at 45° C., 6.17 g of a white solid was obtained. 1.82 g of the product from the previous step (copolymer of HPMA and methacrylic acid-β-Ala-O-succinimide) (14% by weight MA-β-Ala-O-succinimide being equivalent to 1 mmol of the succinimide-activated ester) were added to a N,N-dimethylacetamide solution (15 ml) of H-Phe-Glu(OtBu)-Phe-Lys(Boc)-Phe-Glu(OtBu)-Phe-Lys(Boc)-OEt (SEQ ID NO:98) (1 mmol). The reaction mixture was stirred at 35° C. for 2 h. The reaction was terminated by adding 2 mg 2-aminopropanol. After partial concentration in vacuo, the conjugated polymer is precipitated in 150 ml acetone, filtered, washed once with 50 ml methyl tert-butylether, and finally dried under vacuum at 45° C.

### Example 13

### Synthesis of H₃CO—[(CH₂)₂—O]2—CH₂—CO—NH-FEFKFEFK-NH—[(CH₂)₃—O]₂—(CH₂)₂—NH₂3HCl

To 61.4 g H-Phe-Glu(OtBu)-Phe-Lys(Boc)-Phe-Glu(OtBu)-Phe-Lys(Boc)-NH—(CH₂)₃—O—[(CH₂)2—O]₂—(CH₂)₃—NHBoc (SEQ ID NO:105) (2.7 mmol) in diemthylacetamide, 520 mg 2-[2-(2-methoxyethoxy)-ethoxy]-acetic acid (TODA-OH) (2.7 mmol) was added at room temperature. After cooling to 0° C., 634 mg 1-ethyl-3-(3-dimethylaminopropyl)carbodiimide (3.2 mmol) and 450 mg N-hydroxybenzotriazole (3.2 mmol) were added to the solution. The solution was stirred at 0° C. for an additional 3 h and then allowed to warm to room temperature. After control of the completion of the reaction by HPLC, the reaction mixture was added to a solution of KHSO₄ (0.1 g) in 250 ml water. The resulting precipitate was washed with 75 ml distilled water three times. After drying under vacuum at 45° C., 4.17 g of an off-white solid was obtained. 40 mL of a mixture of trifluoroacetic acid and water (95/5) has added to this solid under stirring. After 30 min stirring at room temperature, the reaction mixture is partially concentrated under vacuum, diluted with CH₂Cl₂ and subsequently concentrated again. This step of concentrating and dilution was repeated several times. The concentrated solution was then poured into 160 ml diisopropylether to obtain a precipitate which was collected. After drying the precipitate under vacuum at 45° C., 8 g of an off-white solid with a n HPLC purity of 84% was obtained. This crude product can optionally be purified further by preparative HPLC using standard methods to obtain a product with a purity of ≧95%.

### Example 14

### Preparation of a Matrix Solution for Drug Delivery

541.1 mg H₃CO—[(CH₂)₂—O]₂—CH₂—CO—NH-FEFKFEFK-NH—[(CH₂)₃—O]₂—(CH₂)₂—NH₂.3HCl was dissolved in 2.49 mL N-methyl pyrrolidinone (NMP) at 40° C. This first matrix solution was cooled to room temperature. 39.8 mg Leuprolide.HCl was weighted into a sterile glass vial, then 1260 μL of the above NMP solution of H₃CO—[(CH₂)₂—O]₂—CH₂—CO—NH-FEFKFEFK-NH—[(CH₂)₃—O]₂—(CH₂)₂—NH₂.3HCl was added. The mixture was homogenized for a few minutes using a vortex, sterile filtered and stored at 4° C. until its final use.

### Example 15

### Coating

### 1. Preparation of the Individual Stock Solutions for Coating

Gamma-irradiated H₂N—[(CH₂)₂—O]₂—CH₂—CO—NH-FEFKFEFK-OEt.TFA (SEQ ID NO: 100) (418 mg) was dispersed in 52 mL sterile HBSS. After vortex mixing and homogenization, the solution was further diluted with 157 mL HBSS. The peptide concentration was controlled by HPLC dosage. Until final use, the solution was stored at 4° C. Before use, the solution was brought back to room temperature and homogenized.
A stock solution of Mpr-hRGDWP-FEFKFEFK-OEt (prepared as described in (WO 2012/045824) was prepared analogously.

### 2. Preparation of the Final Solution for Coating

Under sterile conditions, 13.4 mL of the stock solution of Mpr-hRGDWP-FEFKFEFK-OEt as prepared above and 7.2 mL of the stock solution of H₂N—[(CH₂)₂—O]₂—CH₂—CO—NH-FEFKFEFK-OEt as prepared above were mixed and further diluted with 9.4 mL HBSS. The peptide concentration in the final coating solution was controlled by HPLC dosage. Until its final use, the solution was stored at 4° C. Before use, the solution was brought back to room temperature and homogenized.

### 3. Coating Procedure

Under sterile conditions, 5.25 mL of the final coating solution was introduced into a 25 cm²T-flask (TC-treated, Corning®). The flask was incubated at 37° C. under 5% CO₂ for at least 8 hours. After this incubation time, the coating solution was discarded; the T-flask with rinsed with 5 mL PBS. The flask was ready to be inoculated with cell culture preparation (typically 7 mL).

1. A compound comprising a peptide moiety linked through a linkage to a short-chain polyethylene glycol moiety wherein the short-chain polyethylene glycol moiety comprises equal to or less than 25 oxyethylene moieties.

2. The compound of claim 1, wherein the linkage is a chemical bond or a linear or branched alkylidene linkage, a thioether linkage, a amino linkage, amide linkage, ester linkage or a ether linkage.

3. The compound of claim 1, wherein the short-chain polyethylene glycol moiety comprises one or more functional groups that can be protonated or deprotonated under physiological conditions.

4. The compound according to claim 1, wherein the peptide moiety is able to self-assemble in a β-sheet, a coiled coil ot-helix structure, a peptide triple helix structure, or combinations thereof.

5. The compound according to claim 1, wherein the peptide moiety is an octapeptide moiety formed by the combination of two sequences chosen independently from the group consisting of FEFE (SEQ ID NO:1), FEFK (SEQ ID NO:2), FEFD (SEQ ID NO:3), FEFR (SEQ ID NO:4), FRFR (SEQ ID NO:5), FRFK (SEQ ID NO:6), FRFE (SEQ ID NO:7), FRFD (SEQ ID NO:8), FKFE (SEQ ID NO:9), FKFK (SEQ ID NO: 10), FKFR (SEQ ID NO: 11), FKFD (SEQ ID NO: 12), FDFD (SEQ ID NO: 13), FDFE (SEQ ID NO: 14), FDFR (SEQ ID NO: 15), FDFK (SEQ ID NO: 16), WEWE (SEQ ID NO: 17), WKWK (SEQ ID NO: 18), WRWR (SEQ ID NO: 19), WEWK (SEQ ID NO:20), WKWE (SEQ ID NO:21), WEWR (SEQ ID NO:22), WRWE (SEQ ID NO:23), WKWR (SEQ ID NO:24), WRWK (SEQ ID NO:25), WDWD (SEQ ID NO:26), WDWE (SEQ ID NO:27), WEWD (SEQ ID NO:28), WDWK (SEQ ID NO:29), WKWD (SEQ ID NO:30), WDWR (SEQ ID NO:31), WRWD (SEQ ID NO:32), IEIE (SEQ ID NO:33), IEIK (SEQ ID NO:34), IRIR (SEQ ID NO:35), IKIK (SEQ ID NO:36), IKIE (SEQ ID NO:37), IEIR (SEQ ID NO:38), IRIE (SEQ ID NO:39), IKIR (SEQ ID NO:40), IRIK (SEQ ID NO:41), IDID (SEQ ID NO:42), IDFE (SEQ ID NO:43), IEID (SEQ ID NO:44), IDIK (SEQ ID NO:45), IKID (SEQ ID NO:46), IDIR (SEQ ID NO:47), IRID (SEQ ID NO:48), YEYE (SEQ ID NO:49), YKYK (SEQ ID NO:50), YRYR (SEQ ID NO:51), YEYK (SEQ ID NO:52), YKYE (SEQ ID NO:53), YEYR (SEQ ID NO:54), YRYE (SEQ ID NO:55), YKYR (SEQ ID NO:56), YRYK (SEQ ID NO:57), YDYD (SEQ ID NO:58), YDYE (SEQ ID NO:59), YEYD (SEQ ID NO:60), YDYK (SEQ ID NO:61), YKYD (SEQ ID NO:62), YDYR (SEQ ID NO:63), YRYD (SEQ ID NO:64), Nle-E-Nle-E (SEQ ID NO:65), Nle-K-Nle-K (SEQ ID NO:66), Nle-R-Nle-R (SEQ ID NO:67), Nle-E-Nle-K (SEQ ID NO:68), Nle-K-Nle-E (SEQ ID NO:69), Nle-E-Nle-R (SEQ ID NO:70), Nle-R-Nle-E (SEQ ID NO:71), Nle-K-Nle-R (SEQ ID NO:72), Nle-R-Nle-K (SEQ ID NO:73), Nle-D-Nle-D (SEQ ID NO:74), Nle-D-Nle-E (SEQ ID NO:75), Nle-E-Nle-D (SEQ ID NO:76), Nle-D-Nle-K (SEQ ID NO:77), Nle-K-Nle-D (SEQ ID NO:78), Nle-D-Nle-R (SEQ ID NO:79), and Nle-R-Nle-D (SEQ ID NO:80).

6. The compound according to claim 1, wherein the peptide moiety is selected from the group consisting of FEFKFEFK (SEQ ID NO: 81), FEFEFKFK (SEQ ID NO: 82), FDFKFDFK (SEQ ID NO:83), FDFDFKFK (SEQ ID NO:84), FEFRFEFR (SEQ ID NO:85), FEFEFRFR (SEQ ID NO:86), YDYKYDYK (SEQ ID NO:87), YDYDYKYK (SEQ ID NO:88), YEYRYEYR (SEQ ID NO:89), YEYKYEYK (SEQ ID NO:90), YEYEYKYK (SEQ ID NO:91), WEWKWEWK (SEQ ID NO:92), WEWEWKWK (SEQ ID NO:93), WDWKWDWK (SEQ ID NO:94), and WDWDWKWK (SEQ ID NO:95).

7. The compound according to claim 1, wherein the peptide moiety is FEFKFEFK (SEQ ID NO: 81).

8. A hydrogel comprising the compound according to claim 1.

9. The hydrogel of claim 8 further comprising at least one self-assembling peptide linked through a linkage to a bioactive sequence.

10. The hydrogel of claim 9 further comprising at least one self-assembling peptide linked through a linkage to a polyacrylamide moiety.

11. The hydrogel of claim 10 wherein the at least one self-assembling peptide linked through a linkage to a polyacrylamide residue comprises an oligopeptide moiety comprising alternating hydrophobic and charged amino acids.

12. The hydrogel of claim 10, wherein the polyacrylamide moiety is selected from the group consisting of poly-(N-isopropylacrylamide) (IP A), poly(2-hydroxyethyl methacrylate) (HEMA) and poly[N-(2-hydroxypropyl)methacrylamide] (HPMA).

13. The hydrogel of claim 10, wherein the linkage is a covalent bond, beta-alanine or aminovaleric acid.

14. The process of coating a surface used in cell culture or for drug delivery, comprising coating the surface with a compound according to claim 1 or of a hydrogel comprising said compound or for drug delivery.

15. The process of replacing collagen in cell culture applications comprising replacing collagen in a cell culture application with a compound according to claim 1 or a hydrogel comprising said compound.

16. The hydrogel of claim 9, wherein the bioactive sequence is RGD (Arg-Gly-Asp) or hRGD (Har-Gly-Asp).
